(19) United States
(12) Patent Application Publication
(10) Pub. No.: US 2025/0059600 A1
(43) Pub. Date: Feb. 20, 2025

(54) MASS BASED DETECTION OF PCR AMPLICONS

(71) Applicant: Roche Molecular Systems, Inc., Pleasanton, CA (US)

(72) Inventor: Laura M. Jung, Oakland, CA (US)

(21) Appl. No.: 18/717,650

(22) PCT Filed: Dec. 6, 2022
(86) PCT No.: PCT/EP2022/084628

§ 371 Date: Jun. 7, 2024

Related U.S. Application Data
(60) Provisional application No. 63/287,924, filed on Dec. 9, 2021.

(30) Foreign Application Priority Data
Dec. 6, 2022 (WO) PCT/EP2022/084628

(51) Int. Cl. C12Q 1/6872

(52) U.S. Cl. CPC C12Q 1/6872

(57) ABSTRACT

The present invention provides for novel methods and compositions for nucleic acid sequence detection. Unique, identifying positively charged tags from oligonucleotide probes, bound to target nucleic acids, are produced during PCR by the 5′-nuclease activity of the polymerase. The identity of the targets can be determined by identifying the unique positively charged tags.

### FIELD OF THE INVENTION

The present invention relates to the field of nucleic acid detection. In particular, the present invention provides for a method to perform high throughput multiplex detection of target nucleic acids.

### BACKGROUND OF THE INVENTION

Many methods for detection of target nucleic acids are known. Currently available homogeneous assays for nucleic acid detection include the TaqMan®, Ampliflour®, dye-binding, allele-selective kinetic PCR and Scorpion® primer assays. These assay procedures are not readily multiplexed due to the requirement for a different dye for each target nucleic acid to be detected, and thus are limited in their potential for improvement. To overcome such limitations, several recent studies have disclosed the use of oligonucleotide probes containing a cleavable “tag” portion, which can be readily separated and detected (e.g. see Chenna et al, U.S. Patent Application Publication No. 2005/0053939; Van Den Boom, U.S. Pat. No. 8,133,701). However, the results from these studies show that problems remain in being able to accurately correlate the detection tags with the target nucleic acid and the need still exists for an accurate method to perform high throughput multiplex detection of target nucleic acids.

### SUMMARY OF THE INVENTION

The present invention provides for a novel method for nucleic acid sequence detection, specifically, detection of multiple target nucleic acid sequences in a single polymerase chain reaction (PCR) assay. The invention utilizes standard TaqMan® PCR chemistry, in which the 5′-3′ nuclease activity of a DNA polymerase cleaves a target specific label on a hybridized probe. In this case, the label is a molecule that becomes positively charged under acidic conditions and is detected with positive ion ESI-LCMS (electrospray ionization liquid chromatography mass spectrometer). These Positive Ion Reporter Tags (PIRTs) are small molecules with discrete masses used to identify numerous amplicons similar to how different fluorescent dyes are utilized in TaqMan® technology. The advantage is the facile design of a large number of unique PIRT labels, whereas TaqMan® probe designs are constrained by the limited number of fluorescent dyes available as reporters. The number of distinct molecules that could potentially be used as PIRT labels are so numerous that the number of detectable probes would be unlimited, thus greatly expanding the multiplexing capability.

Therefore in one aspect, the invention provides for a method of detecting the presence or absence of a target nucleic acid sequence in a sample, comprising the steps of (a) preparing a reaction mixture by contacting a sample comprising a target nucleic acid with (i) a pair of oligonucleotide primers comprising a first oligonucleotide primer and a second oligonucleotide primer, wherein the first oligonucleotide primer comprises a sequence complementary to a region in one strand of the target nucleic acid sequence and primes the synthesis of a first extension product, and wherein the second oligonucleotide primer comprises a sequence complementary to a region in said first extension product and primes the synthesis of a nucleic acid strand complementary to said first extension product, and (ii) an oligonucleotide probe comprising a Positive Ion Reporter Tag (PIRT) at the 5′-terminus, whereby the PIRT is protonated under acidic conditions; (b) amplifying said target nucleic acid sequence with a nucleic acid polymerase having 5′ to 3′ nuclease activity under conditions that allows annealing of said pair of oligonucleotide primers and said oligonucleotide probe to the target nucleic acid sequence and synthesis of primer extension products from said pair of oligonucleotide primers, while the 5′ to 3′ nuclease activity of said nucleic acid polymerase is able to cleave and release from the annealed oligonucleotide probe, fragments containing the PIRT with or without additional nucleotides from the oligonucleotide probe; and (c) detecting the presence or absence of the PIRT by an electrospray ionization liquid chromatography mass spectrometer (ESI-LCMS) under acidic conditions, thereby detecting the presence or absence of the target nucleic acid sequence in the sample; wherein no post-amplification sample purification step is performed between step (b) and step (c). In certain embodiments, two or more target nucleic acids are detected in a single multiplexed reaction. In other embodiments, two or more oligonucleotide probes are used to detect the two or more target nucleic acids in the single multiplexed reaction. In some embodiments, the amplifying step is a polymerase chain reaction (PCR) or a real-time polymerase chain reaction (RT-PCR). In some embodiments, the oligonucleotide probe is a TaqMan® probe comprising a Positive Ion Reporter Tag (PIRT) at the 5′-terminus. In yet another embodiment, the detecting step (c) is done by a mass spectrometer, selected from Electrospray Ionization-Time of Flight Mass Spectrometer (ESI-TOF-MS) or Electrospray Ionization-Liquid Chromatography Mass Spectrometer (ESI-LC-MS). In a further embodiment, the nucleic acid polymerase is a thermostable DNA polymerase. In certain embodiments, the PIRT comprises a positive ion entity selected from C6-amino dT, C6-amino, and C12-amino. In yet another embodiment, the PIRT further comprises spacer moieties selected from C3 Spacer, C9 Spacer, C18 Spacer, d Spacer and combinations of the spacer moieties thereof. In another aspect, the invention provides for a composition comprised of an oligonucleotide probe wherein said oligonucleotide probe comprises a Positive Ion Reporter Tag (PIRT) at the 5′-terminus, whereby the PIRT is protonated under acidic conditions. In certain embodiments, the PIRT comprises a positive ion entity selected from C6-amino dT, C6-amino, and C12-amino. In yet another embodiment, the PIRT further comprises spacer moieties selected from C3 Spacer, C9 Spacer, C18 Spacer, d Spacer and combinations of the spacer moieties thereof.

### BRIEF DESCRIPTION OF THE DRAWINGS

FIG. 1 represents an illustrative description of the methods of the present invention.

FIG. 2 (A) The Positive Ion Reporter Tag (PIRT) is designed with a positively charged group, and may include the addition of a mass adjusting subunit. These are coupled to the 5′ end of the oligonucleotide probe during automated DNA synthesis. (B) Examples of positively charged functional groups include an amino group on a phosphorylated 6 or 12 carbon chain (C6 amino, C12 amino) and an amino modified 6 carbon deoxythymidine (C6 amino dT). (C) Various neutral molecules may be added to produce mass distinguishing characteristics. Spacer phosphoramidites are examples of commercially available molecules that meet these requirements.

FIG. 3 HPV TaqMan® probes with PIRT labels as described in Example 5. The HPV probes targeting subtype 33, 16 and 18 were modified with an amino modified 6 carbon deoxythymidine, amino modified 6 carbon chain and 12 carbon chain respectively. The generic internal control (GIC) was modified with an amino modified 6 carbon deoxythymidine plus a 3 carbon spacer as a mass adjuster.

FIG. 4 The PIRT tag is liberated by the 5-3′ exonuclease activity of the DNA polymerase during PCR amplification of the targeted sequence. The cleaved fragment may include a few of the terminal nucleic acid bases from the probe depending on the polymerase cleavage efficiency. (A) The HPV 16 probe was designed with an amino label on a 6 carbon chain and attached with a phosphodiester linkage to the 5′ end of the oligonucleotide probe. The cleavage fragment is detected by liquid chromatography mass spectrometry (LCMS) as a peak with an exact mass-to-charge (m/z) of 711.2148 in the mass spectrum (B). This is consistent with a protonated (+H) form of the PIRT label plus the two terminal bases from the 5′-end of the probe with a calculated exact m/z of 711.2156. (C) The m/z of the protonated cleavage fragment is plotted against retention time in construction of an extracted ion chromatogram (EIC). The presence of an EIC peak and its retention time are used to identify the amplification of the target nucleic acid.

FIG. 5 The m/z of the most abundant fragment is determined in a PCR single-plex assay for each of the probes. This m/z and retention time is subsequently used as the target reporter for the detection of a specific target in a PCR multiplex assay.

FIG. 6 Extracted ion chromatograms (EIC) are constructed for each of the 4 target masses as described in Example 7 with 100 copies of HPV 16 and GIC gBlock templates. The presence of a peak indicates the positive detection of the corresponding target.

FIG. 7 Extracted ion chromatograms (EIC) are constructed for each of the 4 target masses as described in Example 7 with 100 copies of HPV 18 and GIC gBlock templates. The presence of a peak indicates the positive detection of the corresponding target.

FIG. 8 Extracted ion chromatograms (EIC) are constructed for each of the 4 target masses as described in Example 7 with 100 copies of HPV 33 and GIC gBlock templates. The presence of a peak indicates the positive detection of the corresponding target. FIG. 9 Embodiments of Positive Ion Reporter Tags (PIRTs) with distinguishable masses.

### DETAILED DESCRIPTION OF THE INVENTION

### Definitions

The term “sample” as used herein includes a specimen or culture (e.g., microbiological cultures) that includes nucleic acids. The term “sample” is also meant to include both biological and environmental samples. A sample may include a specimen of synthetic origin. Biological samples include whole blood, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), biopsy sample, urine, feces, sputum, saliva, nasal mucous, prostate fluid, semen, lymphatic fluid, bile, tears, sweat, breast milk, breast fluid, embryonic cells and fetal cells. In a preferred embodiment, the biological sample is blood, and more preferably plasma. As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.
The terms “target” or “target nucleic acid” as used herein are intended to mean any molecule whose presence is to be detected or measured or whose function, interactions or properties are to be studied. Therefore, a target includes essentially any molecule for which a detectable probe (e.g., oligonucleotide probe) or assay exists, or can be produced by one skilled in the art. For example, a target may be a biomolecule, such as a nucleic acid molecule, a polypeptide, a lipid, or a carbohydrate, that is capable of binding with or otherwise coming in contact with a detectable probe (e.g., an antibody), wherein the detectable probe also comprises nucleic acids capable of being detected by methods of the invention. As used herein, “detectable probe” refers to any molecule or agent capable of hybridizing or annealing to a target biomolecule of interest and allows for the specific detection of the target biomolecule as described herein. In one aspect of the invention, the target is a nucleic acid, and the detectable probe is an oligonucleotide. The terms “nucleic acid” and “nucleic acid molecule” may be used interchangeably throughout the disclosure. The terms refer to oligonucleotides, oligos, polynucleotides, deoxyribonucleotide (DNA), genomic DNA, mitochondrial DNA (mtDNA), complementary DNA (cDNA), bacterial DNA, viral DNA, viral RNA, RNA, message RNA (mRNA), transfer RNA (tRNA), ribosomal RNA (rRNA), siRNA, catalytic RNA, clones, plasmids, M13, P1, cosmid, bacteria artificial chromosome (BAC), yeast artificial chromosome (YAC), amplified nucleic acid, amplicon, PCR product and other types of amplified nucleic acid, RNA/DNA hybrids and polyamide nucleic acids (PNAs), all of which can be in either single- or double-stranded form, and unless otherwise limited, would encompass known analogs of natural nucleotides that can function in a similar manner as naturally occurring nucleotides and combinations and/or mixtures thereof. Thus, the term “nucleotides” refers to both naturally-occurring and modified/non-naturally-occurring nucleotides, including nucleoside tri-, di-, and monophosphates as well as monophosphate monomers present within polynucleic acid or oligonucleotide. A nucleotide may also be a ribo-, 2′-deoxy-, 2′,3′-deoxynucleotide as well as a vast array of other nucleotide mimics that are well-known in the art. Mimics include chain-terminating nucleotides, such as 3′-O-methyl, halogenated base or sugar substitutions; alternative sugar structures including non-sugar, alkyl ring structures; alternative bases including inosine; deaza-modified; chi and psi, linker-modified; mass label-modified; phosphodiester modifications or replacements including phosphorothioate, methylphosphonate, boranophosphate, amide, ester, ether; and a basic or complete internucleotide replacements, including cleavage linkages such a photocleavable nitrophenyl moieties.

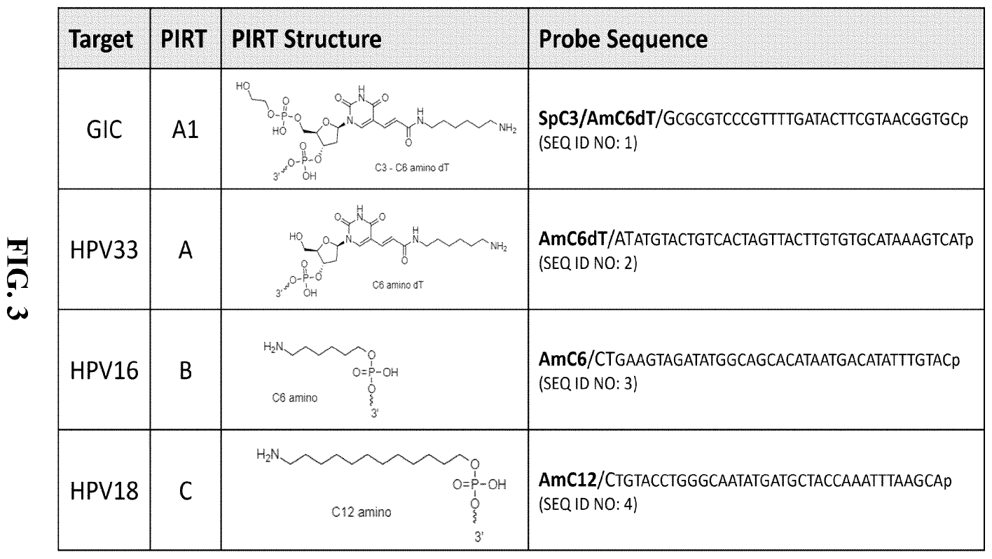

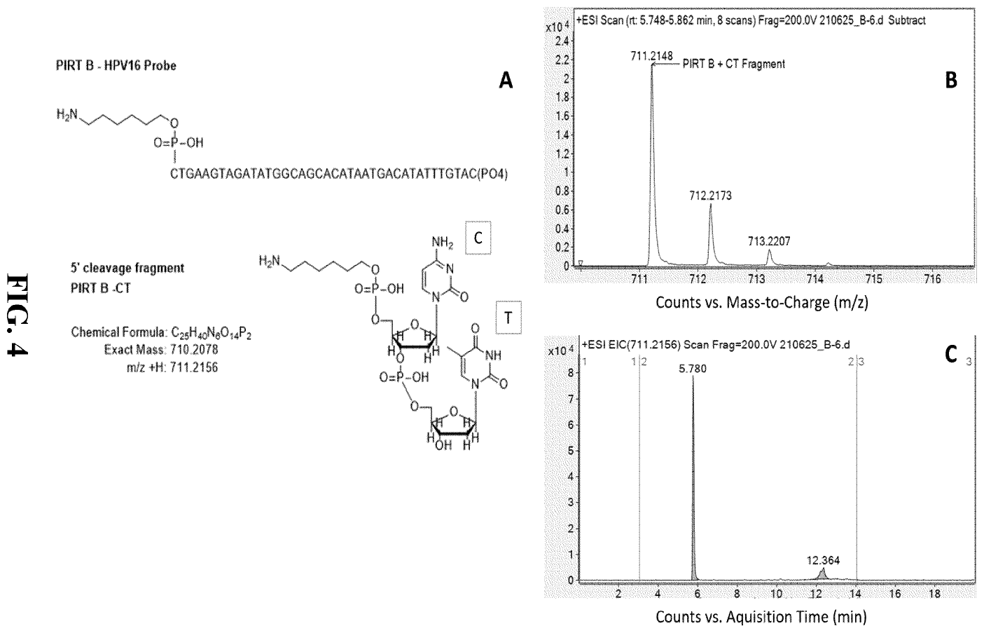

The presence or absence of a target can be measured quantitatively or qualitatively. Targets can come in a variety of different forms including, for example, simple or complex mixtures, or in substantially purified forms. For example, a target can be part of a sample that contains other components or can be the sole or major component of the sample. Therefore, a target can be a component of a whole cell or tissue, a cell or tissue extract, a fractionated lysate thereof or a substantially purified molecule. Also, a target can have either a known or unknown sequence or structure.
The term “amplification reaction” refers to any in vitro means for multiplying the copies of a target sequence of nucleic acid.
“Amplifying” refers to a step of submitting a solution to conditions sufficient to allow for amplification. Components of an amplification reaction may include, but are not limited to, e.g., primers, a polynucleotide template, polymerase, nucleotides, dNTPs and the like. The term “amplifying” typically refers to an “exponential” increase in target nucleic acid. However, “amplifying” as used herein can also refer to linear increases in the numbers of a select target sequence of nucleic acid, but is different from a one-time, single primer extension step.
“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990.
“Oligonucleotide” as used herein refers to linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target nucleic acid. Usually monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG,” it will be understood that the nucleotides are in 5′-3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. Where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill.
As used herein “oligonucleotide primer”, or simply “primer”, refers to a polynucleotide sequence that hybridizes to a sequence on a target nucleic acid template and facilitates the detection of an oligonucleotide probe. In amplification embodiments of the invention, an oligonucleotide primer serves as a point of initiation of nucleic acid synthesis. In non-amplification embodiments, an oligonucleotide primer may be used to create a structure that is capable of being cleaved by a cleavage agent. Primers can be of a variety of lengths and are often less than 50 nucleotides in length, for example 12-25 nucleotides, in length. The length and sequences of primers for use in PCR can be designed based on principles known to those of skill in the art.
The term “oligonucleotide probe” as used herein refers to a polynucleotide sequence capable of hybridizing or annealing to a target nucleic acid of interest and allows for the specific detection of the target nucleic acid.
A “mismatched nucleotide” or a “mismatch” refers to a nucleotide that is not complementary to the target sequence at that position or positions. An oligonucleotide probe may have at least one mismatch, but can also have 2, 3, 4, 5, 6 or 7 or more mismatched nucleotides.
The term “polymorphism” as used herein refers to an allelic variant. Polymorphisms can include single nucleotide polymorphisms (SNP's) as well as simple sequence length polymorphisms. A polymorphism can be due to one or more nucleotide substitutions at one allele in comparison to another allele or can be due to an insertion or deletion, duplication, inversion and other alterations known to the art.
The term “Positive Ion Reporter Tags” or “PIRTs” are small positively charged molecules with discrete masses that are used to identify numerous amplification products of target nucleic acids. Discrete masses are achieved by adjusting the mass of each PIRT with the addition of neutral charged entities such as linkers or spacer arms of various sizes and compositions. Embodiments of PIRTs are shown on FIGS. 2 and 3. Examples of positively charged functional groups include a phosphorylated C3, C6, C9, C12, or C18 carbon chain attached to an amino group (see FIG. 2B exemplifying C6-amino and C12-amino moieties) or a nucleotide (such as dT, dU, dG, dC, dA or a nucleotide analog thereof) attached with an amino modified C3, C6, C9, C12, or C18 carbon chain (see FIG. 2B exemplifying a C6-amino-deoxythymidine (dT) moiety).
“Positive Ion Reporter Tag probes” are labeled on the 5′-end with a chemical modification, such as an amino group, that is protonated under acidic conditions. Preferred acidic buffers include but are not limited to formic acid, acetic acid and trifluoroacetic acid that have a pH of less than 4.0, preferably between pH 2.0 and pH 4.0. Amine-modified probes can typically be generated during oligonucleotide synthesis whereby the amino group is attached to the 5′-terminus nucleotide via linkage to carbon chains that have various lengths. Such carbon chains are available as C3, C6, C9, C12 and C18 spacer phosphoramidites or d spacers and are commercially available (for example at Glen Research, Sterling, VA and Integrated DNA Technologies, Inc. Coralville, IA). The compounds may also be added in multiple additions when a longer spacer is required. Furthermore, additional positive ion tags having unique masses can be generated by adjusting the mass with the addition of spacers as neutral entities. Non-limiting examples of Positive Ion Reporter Tags are shown in FIG. 9.
The term “modification” as used herein refers to alterations of the oligonucleotide probe at the molecular level (e.g., base moiety, sugar moiety or phosphate backbone). Nucleoside modifications include, but are not limited to, the introduction of cleavage blockers or cleavage inducers, the introduction of minor groove binders, isotopic enrichment, isotopic depletion, the introduction of deuterium, and halogen modifications. Nucleoside modifications may also include moieties that increase the stringency of hybridization or increase the melting temperature of the oligonucleotide probe. For example, a nucleotide molecule may be modified with an extra bridge connecting the 2′ and 4′ carbons resulting in locked nucleic acid (LNA) nucleotide that is resistant to cleavage by a nuclease.
The term “specific” or “specificity” in reference to the binding of one molecule to another molecule, such as a probe for a target polynucleotide, refers to the recognition, contact, and formation of a stable complex between the two molecules, together with substantially less recognition, contact, or complex formation of that molecule with other molecules. As used herein, the term “anneal” refers to the formation of a stable complex between two molecules.

A probe is “capable of annealing” to a nucleic acid sequence if at least one region of the probe shares substantial sequence identity with at least one region of the complement of the nucleic acid sequence. “Substantial sequence identity” is a sequence identity of at least about 80%, preferably at least about 85%, more preferably at least about 90%, 95% or 99%, and most preferably 100%. For the purpose of determining sequence identity of a DNA sequence and a RNA sequence, U and T often are considered the same nucleotide. For example, a probe comprising the sequence ATCAGC is capable of hybridizing to a target RNA sequence comprising the sequence GCUGAU.
The term “cleavage agent” as used herein refers to any means that is capable of cleaving an oligonucleotide probe to yield fragments of mass-distinguishable sizes, including but not limited to enzymes. For methods wherein amplification does not occur, the cleavage agent may serve solely to cleave, degrade or otherwise release the second portion of the oligonucleotide probe or fragments thereof. The cleavage agent may be an enzyme. The cleavage agent may be natural, synthetic, unmodified or modified.

For methods wherein amplification occurs, the cleavage agent is preferably an enzyme that possess synthetic (or polymerization) activity and nuclease activity. Such an enzyme is often a nucleic acid amplification enzyme. An example of a nucleic acid amplification enzyme is a nucleic acid polymerase enzyme such as Thermus aquaticus (Taq) DNA polymerase or E. coli DNA polymerase I. The enzyme may be naturally occurring, unmodified or modified.
The term “cleaves said fragments up to the exonuclease-resistant modification” means a cleavage activity that would cleave the fragments until reaching the exonuclease-resistant modification itself or at a defined nucleotide located proximal to the exonuclease-resistant modification. For a 3′ to 5′ exonuclease activity, the defined nucleotide proximal to the modification could be located at the first position immediately 3′from the modification. Alternatively, the defined nucleotide could be located two or three or even more positions 3′ from the modification, so long as cleavage by the 3′ to 5′ exonuclease consistently terminates at the position of the defined nucleotide.
A “nucleic acid polymerase” refers to an enzyme that catalyzes the incorporation of nucleotides into a nucleic acid. Exemplary nucleic acid polymerases include DNA polymerases, RNA polymerases, terminal transferases, reverse transcriptases, telomerases and the like.
A “thermostable DNA polymerase” refers to a DNA polymerase that is stable (i.e., resists breakdown or denaturation) and retains sufficient catalytic activity when subjected to elevated temperatures for selected periods of time. For example, a thermostable DNA polymerase retains sufficient activity to effect subsequent primer extension reactions, when subjected to elevated temperatures for the time necessary to denature double-stranded nucleic acids. Heating conditions necessary for nucleic acid denaturation are well known in the art and are exemplified in U.S. Pat. Nos. 4,683,202 and 4,683,195. As used herein, a thermostable polymerase is typically suitable for use in a temperature cycling reaction such as the polymerase chain reaction (“PCR”). The examples of thermostable nucleic acid polymerases include Thermus aquaticus Taq DNA polymerase, Thermus sp. Z05 polymerase, Thermus flavus polymerase, Thermotoga maritima polymerases, such as TMA-25 and TMA-30 polymerases, Tth DNA polymerase, and the like.
A “modified” polymerase refers to a polymerase in which at least one monomer differs from the reference sequence, such as a native or wild-type form of the polymerase or another modified form of the polymerase. Exemplary modifications include monomer insertions, deletions, and substitutions. Modified polymerases also include chimeric polymerases that have identifiable component sequences (e.g., structural or functional domains, etc.) derived from two or more parents. Also included within the definition of modified polymerases are those comprising chemical modifications of the reference sequence. The examples of modified polymerases include G46E E678G CS5 DNA polymerase, G46E L329A E678G CS5 DNA polymerase, G46E L329A D640G S671F CS5 DNA polymerase, G46E L329A D640G S671F E678G CS5 DNA polymerase, a G46E E678G CS6 DNA polymerase, Z05 DNA polymerase, ΔZ05 polymerase, ΔZ05-Gold polymerase, ΔZ05R polymerase, E615G Taq DNA polymerase, E678G TMA-25 polymerase, E678G TMA-30 polymerase, and the like.
The term “5′ to 3′ nuclease activity” or “5′-3′ nuclease activity” refers to an activity of a nucleic acid polymerase, typically associated with the nucleic acid strand synthesis, whereby nucleotides are removed from the 5′ end of nucleic acid strand, e.g., E. coli DNA polymerase I has this activity, whereas the Klenow fragment does not. Some enzymes that have 5′ to 3′ nuclease activity are 5′ to 3′ exonucleases. The term “single strand-specific 5′-3′ exonuclease” refers to exonucleases acting from the 5′ end with a preference for single stranded nucleic acids over double stranded nucleic acids. Examples of such single strand—specific 5′-3′ exonucleases include Exonuclease from B. subtilis, Phosphodiesterase from spleen, Exonuclease II from yeast, Exonuclease V from yeast, and Exonuclease from Neurospora crassa.

Various aspects of the present invention are based on a special property of nucleic acid polymerases. Nucleic acid polymerases can possess several activities, among them, a 5′ to 3′ nuclease activity whereby the nucleic acid polymerase can cleave mononucleotides or small oligonucleotides from an oligonucleotide annealed to its larger, complementary polynucleotide. In order for cleavage to occur efficiently, an upstream oligonucleotide must also be annealed to the same larger polynucleotide.

The detection of a target nucleic acid utilizing the 5′ to 3′ nuclease activity can be performed by a “TaqMan®” or “5′-nuclease assay”, as described in U.S. Pat. Nos. 5,210,015; 5,487,972; and 5,804,375; and Holland et al., 1988, Proc. Natl. Acad. Sci. USA 88:7276-7280, all incorporated by reference herein. In the TaqMan® assay, labeled detection probes that hybridize within the amplified region are present during the amplification reaction. The probes are modified so as to prevent the probes from acting as primers for DNA synthesis. The amplification is performed using a DNA polymerase having 5′ to 3′ nuclease activity on double stranded nucleic acids. During each synthesis step of the amplification, any probe which hybridizes to the target nucleic acid downstream from the primer being extended is degraded by the 5′ to 3′ nuclease activity of the DNA polymerase. Thus, the synthesis of a new target strand also results in the degradation of a probe, and the accumulation of degradation product provides a measure of the synthesis of target sequences.

Any method suitable for detecting degradation product can be used in a 5′ nuclease assay. Often, the detection probe is labeled with two fluorescent dyes, one of which is capable of quenching the fluorescence of the other dye. The dyes are attached to the probe, typically with the reporter or detector dye attached to the 5′-terminus and the quenching dye attached to an internal site, such that quenching occurs when the probe is in an unhybridized state and such that cleavage of the probe by the 5′ to 3′ nuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated by reference herein, describe alternative methods for detecting the degradation of probe, which occurs concomitant with amplification.

A 5′ nuclease assay for the detection of a target nucleic acid can employ any polymerase that has a 5′ to 3′ nuclease activity. Thus, in some embodiments, the polymerases with 5′-nuclease activity are thermostable and thermoactive nucleic acid polymerases. Such thermostable polymerases include, but are not limited to, native and recombinant forms of polymerases from a variety of species of the eubacterial genera Thermus, Thermatoga, and Thermosipho, as well as chimeric forms thereof For example, Thermus species polymerases that can be used in the methods of the invention include Thermus aquaticus (Taq) DNA polymerase, Thermus thermophilus (Tth) DNA polymerase, Thermus species Z05 (Z05) DNA polymerase, Thermus species sps17 (sps17), and Thermus species Z05 (e.g., described in U.S. Pat. Nos. 5,405,774; 5,352,600; 5,079,352; 4,889,818; 5,466,591; 5,618,711; 5,674,738, and 5,795,762. Thermatoga polymerases that can be used in the methods of the invention include, for example, Thermatoga maritima DNA polymerase and Thermatoga neapolitana DNA polymerase, while an example of a Thermosipho polymerase that can be used is Thermosipho africanus DNA polymerase. The sequences of Thermatoga maritima and Thermosipho africanus DNA polymerases are published in International Patent Application No. PCT/US91/07035 with Publication No. WO 92/06200. The sequence of Thermatoga neapolitana may be found in International Patent Publication No. WO 97/09451. In the 5′ nuclease assay, the amplification detection is typically concurrent with amplification (i.e., “real-time”). In some embodiments, the amplification detection is quantitative, and the amplification detection is real-time. In some embodiments, the amplification detection is qualitative (e.g., end-point detection of the presence or absence of a target nucleic acid). In some embodiments, the amplification detection is subsequent to amplification. In some embodiments, the amplification detection is qualitative, and the amplification detection is subsequent to amplification.

The present invention provides for oligonucleotide primers and probes. It is not intended that the methods used to produce these probes and primers be in any way limited. One of skill in the art is well familiar with the wide variety of chemical synthesis strategies and reagents for producing probes and primers. It is also not intended that the oligonucleotide probes of the invention be limited to limited to naturally occurring nucleotide structures or naturally occurring bases (e.g., adenine, guanine, thymine, cytosine, and uracil). In addition to the naturally occurring heterocyclic bases that are typically found in nucleic acids, non-natural nucleic acid analogs also find use with the invention.

Non-natural analogs include those having non-naturally occurring heterocyclic or other modified bases. In particular, many non-naturally occurring bases are described further in, e.g., Seela et al. (1991) Helv. Chim. Acta 74:1790, Grein et al. (1994) Bioorg. Med. Chem. Lett. 4:971-976, and Seela et al. (1999) Helv. Chim. Acta 82:1640. To further illustrate, certain bases used in nucleotides that act as melting temperature (Tm) modifiers are optionally included. For example, some of these include 7-deazapurines (e.g., 7-deazaguanine, 7-deazaadenine, etc.), pyrazolo [3,4-d]pyrimidines, propynyl-dN (e.g., propynyl-dU, propynyl-dC, etc.), and the like. See, e.g., U.S. Pat. No. 5,990,303, entitled “SYNTHESIS OF 7-DEAZA-2′-DEOXYGUANOSINE NUCLEOTIDES,” which issued Nov. 23, 1999 to Seela. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytosine; 5-fluorocytosine; 5-chlorocytosine; 5-iodocytosine; 5-bromocytosine; 5-methylcytosine; 5-propynylcytosine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, 4-acetylcytosine, 5-(carboxyhydroxy-methyl) uracil, 5 carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 7-deazaadenine, 2-methyladenine, 2-methylguanine, 3-methylcytosine, 5-methylcytosine, N6-methyladenine, 7-methylguanine, 7-deazaguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2 thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3) w, 2,6-diaminopurine, and 5-propynyl pyrimidine, and the like. To further illustrate, other examples of modified oligonucleotides include those having one or more locked nucleid acid (LNA™) monomers (oligonucleotides comprising LNA™ monomers available from, e.g., Link Technologies, Ltd., Lanarkshire, Scotland; under license from Exiqon A/S, Vedbæk, Denmark). Nucleotide analogs such as these are also described in, e.g., U.S. Pat. No. 6,639,059, entitled “SYNTHESIS OF [2.2.1] BICYCLO NUCLEOSIDES,” issued Oct. 28, 2003 to Kochkine et al., U.S. Pat. No. 6,303,315, entitled “ONE STEP SAMPLE PREPARATION AND DETECTION OF NUCLEIC ACIDS IN COMPLEX BIOLOGICAL SAMPLES,” issued Oct. 16, 2001 to Skouv, and U.S. Pat. Application Pub. No. 2003/0092905, entitled “SYNTHESIS OF [2.2.1] BICYCLO NUCLEOSIDES,” by Kochkine et al. that published May 15, 2003, which are each incorporated by reference.

Oligonucleotide probes and primers can be prepared using any technique known in the art. In certain embodiments, for example, the oligonucleotide probes and primers are synthesized chemically using any nucleic acid synthesis method, including, e.g., according to the solid phase phosphoramidite method described by Beaucage and Caruthers (1981) Tetrahedron Letts. 22(20):1859 1862, which is incorporated by reference. To further illustrate, oligonucleotides can also be synthesized using a triester method (see, e.g., Capaldi et al. (2000) “Highly efficient solid phase synthesis of oligonucleotide analogs containing phosphorodithioate linkages” Nucleic Acids Res. 28(9):e40 and Eldrup et al. (1994) “Preparation of oligodeoxyribonucleoside phosphorodithioates by a triester method” Nucleic Acids Res. 22(10):1797-1804, which are both incorporated by reference). Other synthesis techniques known in the art can also be utilized, including, e.g., using an automated synthesizer, as described in Needham VanDevanter et al. (1984) Nucleic Acids Res. 12:6159 6168, which is incorporated by reference. A wide variety of equipment is commercially available for automated oligonucleotide synthesis. Multi-nucleotide synthesis approaches (e.g., tri-nucleotide synthesis, etc.) are also optionally utilized. Moreover, the primer nucleic acids optionally include various modifications. In certain embodiments, for example, primers include restriction site linkers, e.g., to facilitate subsequent amplicon cloning or the like. To further illustrate, primers are also optionally modified to improve the specificity of amplification reactions as described in, e.g., U.S. Pat. No. 6,001,611, entitled “MODIFIED NUCLEIC ACID AMPLIFICATION PRIMERS,” issued Dec. 14, 1999 to Will, which is incorporated by reference. Primers and probes can also be synthesized with various other modifications as described herein or as otherwise known in the art.

Probes utilized in the reaction mixtures, methods, and other aspects of the invention have tags containing a positive ion entity. Such tags can be attached to oligonucleotides directly or indirectly by a variety of techniques known in the art. To illustrate, depending on the type of tag used, the tag can be attached to a terminal (5′ or 3′ end of an oligonucleotide primer and/or probe) or a non-terminal nucleotide, and can be attached indirectly through linkers or spacer arms of various sizes and compositions. Using commercially available phosphoramidite reagents, one can produce oligonucleotides containing functional groups (e.g., thiols or primary amines) at either the 5′- or 3′-terminus via an appropriately protected phosphoramidite, and can attach tags to such oligonucleotides using protocols described in, e.g., Innis et al. (Eds.) PCR Protocols: A Guide to Methods and Applications, Elsevier Science & Technology Books (1990)(Innis), which is incorporated by reference.

Essentially any nucleic acid (standard or non-standard, labeled or non-labeled) can be custom or standard ordered from any of a variety of commercial sources, such as The Midland Certified Reagent Company (Midland, TX), Operon Technologies Inc. (Huntsville, AL), Proligo LLC (Boulder, CO), and many others.

“Positive Ion Reporter Tags” (PIRTs) with discrete sizes are distinguished by a particular physical attribute or detection feature, including but not limited to length, mass, charge, or charge-to-mass ratio. In a preferred embodiment, the detection feature is mass. In another related embodiment, the PIRT may be distinguished by a behavior that is related to a physical attribute, including but not limited to mass, time of flight in MALDI-TOF mass spectrometry

Mass spectrometry is the preferred method to detect PIRTs of the invention and thus identify and/or quantitate target nucleic acids. PIRTs can be ionized in a mass spectrometer and the ions separated in space or time based on their mass-to-charge ratio. The mass spectrometer then calculates a mass associated with each ion. Therefore, when referring to mass spectrometry, the term mass can be used for simplicity to describe a mass-to-charge ratio.

Mass spectrometry is a sensitive and accurate technique for separating and identifying molecules. Generally, mass spectrometers have two main components, an ion source for the production of ions and a mass-selective analyzer for measuring the mass-to-charge ratio of ions, which is and converted into a measurement of mass for these ions. Several ionization methods are known in the art and described herein. A PIRT can be charged prior to, during or after cleavage from the oligonucleotide probe. Consequently, a PIRT that will be measured by mass spectrometry does not always require a charge since a charge can be acquired through the mass spectrometry procedure. In mass spectrometry analysis, optional components of a PIRT such as charge and detection moieties can be used to contribute mass to the PIRT.

Different mass spectrometry methods, for example, quadrupole mass spectrometry, ion trap mass spectrometry, time-of-flight mass spectrometry, gas chromatography mass spectrometry and tandem mass spectrometry, as described herein, can utilize various combinations of ion sources and mass analyzers which allows for flexibility in designing customized detection protocols. In addition, mass spectrometers can be programmed to transmit all ions from the ion source into the mass spectrometer either sequentially or at the same time. Furthermore, a mass spectrometer can be programmed to select ions of a particular mass for transmission into the mass spectrometer while blocking other ions.

The ability to precisely control the movement of ions in a mass spectrometer allows for greater options in detection protocols which can be advantageous when a large number of PIRTs, for example, from a multiplex experiment, are being analyzed. For example, in a multiplex experiment with a large number of PIRTs it can be advantageous to select individual reporters from a group of similar reporters and then analyze that reporter separately. Another advantage based on controlling the mass range detected by the mass spectrometer includes the ability to exclude un-cleaved or partially cleaved PIRT-tagged probes from being analyzed, which reduces background noise from the assay.

Mass spectrometers can resolve ions with small mass differences and measure the mass of ions with a high degree of accuracy. Therefore, PIRTs of similar masses can be used together in the same experiment since the mass spectrometer can differentiate the mass of even closely related tags. The high degree of resolution and mass accuracy achieved using mass spectrometry methods allows the use of large sets of tagged probes because the resulting reporter tags can be distinguished from each other. The ability to use large sets of tagged probes is an advantage when designing multiplex experiments.

Another advantage of using mass spectrometry for detecting the mass of a PIRT is based on the high sensitivity of this type of mass analysis. Mass spectrometers achieve high sensitivity by utilizing a large portion of the ions that are formed by the ion source and efficiently transmitting these ions through the mass analyzer to the detector. Because of this high level of sensitivity, even limited amounts of sample can be measured using mass spectrometry. This can be an advantage in a multiplex experiment where the amount of each PIRT species may be small.

Mass spectrometry methods are well known in the art (see Burlingame et al. Anal. Chem. 70:647R-716R (1998); Kinter and Sherman, Protein Sequencing and Identification Using Tandem Mass Spectrometry Wiley-Interscience, New York (2000)). The basic processes associated with a mass spectrometry method are the generation of gas-phase ions derived from the sample, and the measurement of their mass.

The movement of gas-phase ions can be precisely controlled using electromagnetic fields generated in the mass spectrometer. The movement of ions in these electromagnetic fields is proportional to the m/z of the ion and this forms the basis of measuring the m/z and therefore the mass of a sample. The movement of ions in these electromagnetic fields allows the ions to be contained and focused which accounts for the high sensitivity of mass spectrometry. During the course of m/z measurement, ions are transmitted with high efficiency to particle detectors that record the arrival of these ions. The quantity of ions at each m/z is demonstrated by peaks on a graph where the x axis is m/z and the y axis is relative abundance. Different mass spectrometers have different levels of resolution, that is, the ability to resolve peaks between ions closely related in mass. The resolution is defined as R=m/delta m, where m is the ion mass and delta m is the difference in mass between two peaks in a mass spectrum. For example, a mass spectrometer with a resolution of 1000 can resolve an ion with a m/z of 100.0 from an ion with a m/z of 100.1. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Exemplary mass analyzers include a quadrupole mass filter, ion trap mass analyzer and time-of-flight mass analyzer.

The ion formation process is a starting point for mass spectrum analysis. Several ionization methods are available and the choice of ionization method depends on the sample to be analyzed. For example, for the analysis of polypeptides a relatively gentle ionization procedure such as electrospray ionization (ESI) can be desirable. For ESI, a solution containing the sample is passed through a fine needle at high potential which creates a strong electrical field resulting in a fine spray of highly charged droplets that is directed into the mass spectrometer. Other ionization procedures include, for example, fast-atom bombardment (FAB) which uses a high-energy beam of neutral atoms to strike a solid sample causing desorption and ionization. Matrix-assisted laser desorption ionization (MALDI) is a method in which a laser pulse is used to strike a sample that has been crystallized in an UV-absorbing compound matrix. Other ionization procedures known in the art include, for example, plasma and glow discharge, plasma desorption ionization, resonance ionization, and secondary ionization. A PIRT can become ionized prior to, during, or after cleavage from the tagged probe.

Electrospray ionization (ESI) has several properties that are useful for the invention described herein. For example, ESI can be used for biological molecules such as polypeptides that are difficult to ionize or vaporize. In addition, the efficiency of ESI can be very high which provides the basis for highly sensitive measurements. Furthermore, ESI produces charged molecules from solution, which is convenient for analyzing PIRTs that are in solution. In contrast, ionization procedures such as MALDI require crystallization of the sample prior to ionization.

Since ESI can produce charged molecules directly from solution, it is compatible with samples from liquid chromatography systems. For example, a mass spectrometer can have an inlet for a liquid chromatography system, such as an HPLC, so that fractions flow from the chromatography column into the mass spectrometer. This in-line arrangement of a liquid chromatography system and mass spectrometer is sometimes referred to as LC-MS. A LC-MS system can be used, for example, to separate un-cleaved or partially cleaved PIRTs from cleaved PIRTs before mass spectrometry analysis. In addition, chromatography can be used to remove salts or other buffer components from the PCR sample before mass spectrometry analysis. For example, desalting of a sample using a reversed-phase HPLC column, in-line or off-line, can be used to increase the efficiency of the ionization process and thus improve sensitivity of detection by mass spectrometry.

A variety of mass analyzers are available that can be paired with different ion sources. Different mass analyzers have different advantages as known to one skilled in the art and as described herein. The mass spectrometer and methods chosen for detection depends on the particular assay, for example, a more sensitive mass analyzer can be used when a small amount of ions are generated for detection. Several types of mass analyzers and mass spectrometry methods are described below.

Ion mobility mass (IM) spectrometry is a gas-phase separation method that adds new dimensions to mass spectrometry (MS). IM separates gas-phase ions based on their collision cross-section and can be coupled with time-of-flight (TOF) mass spectrometry to yield a powerful tool used in the identification and characterization of proteins and peptides. IM-MS is discussed in more detail by Verbeck et al. in the Journal of Biomolecular Techniques (Vol 13, Issue 2, 56-61).

Quadrupole mass spectrometry utilizes a quadrupole mass filter or analyzer. This type of mass analyzer is composed of four rods arranged as two sets of two electrically connected rods. A combination of rf and dc voltages are applied to each pair of rods which produces fields that cause an oscillating movement of the ions as they move from the beginning of the mass filter to the end. The result of these fields is the production of a high-pass mass filter in one pair of rods and a low-pass filter in the other pair of rods. Overlap between the high-pass and low-pass filter leaves a defined m/z that can pass both filters and traverse the length of the quadrupole. This m/z is selected and remains stable in the quadrupole mass filter while all other m/z have unstable trajectories and do not remain in the mass filter. A mass spectrum results by ramping the applied fields such that an increasing m/z is selected to pass through the mass filter and reach the detector. In addition, quadrupoles can also be set up to contain and transmit ions of all m/z by applying a rf-only field. This allows quadrupoles to function as a lens or focusing system in regions of the mass spectrometer where ion transmission is needed without mass filtering. This will be of use in tandem mass spectrometry as described further below.

A quadrupole mass analyzer, as well as the other mass analyzers described herein, can be programmed to analyze a defined m/z or mass range. This property of mass spectrometers is useful for the invention described herein. Since the mass range of cleaved PIRT will be known prior to an assay, a mass spectrometer can be programmed to transmit ions of the projected correct mass range while excluding ions of a higher or lower mass range. The ability to select a mass range can decrease the background noise in the assay and thus increase the signal-to-noise ratio. In addition, a defined mass range can be used to exclude analysis of any un-cleaved oligonucleotide probes, which would be of higher mass than the mass of the PIRTs. Therefore, the mass spectrometer can accomplish an inherent separation step as well as detection and identification of the PIRTs. Ion trap mass spectrometry utilizes an ion trap mass analyzer. In these mass analyzers, fields are applied so that ions of all m/z are initially trapped and oscillate in the mass analyzer. Ions enter the ion trap from the ion source through a focusing device such as an octapole lens system. Ion trapping takes place in the trapping region before excitation and ejection through an electrode to the detector. Mass analysis is accomplished by sequentially applying voltages that increase the amplitude of the oscillations in a way that ejects ions of increasing m/z out of the trap and into the detector. In contrast to quadrupole mass spectrometry, all ions are retained in the fields of the mass analyzer except those with the selected m/z. One advantage to ion traps is that they have very high sensitivity, as long as one is careful to limit the number of ions being tapped at one time. Control of the number of ions can be accomplished by varying the time over which ions are injected into the trap. The mass resolution of ion traps is similar to that of quadrupole mass filters, although ion traps do have low m/z limitations.

Time-of-flight mass spectrometry utilizes a time-of-flight mass analyzer. For this method of m/z analysis, an ion is first given a fixed amount of kinetic energy by acceleration in an electric field (generated by high voltage). Following acceleration, the ion enters a field-free or “drift” region where it travels at a velocity that is inversely proportional to its m/z. Therefore, ions with low m/z travel more rapidly than ions with high m/z. The time required for ions to travel the length of the field-free region is measured and used to calculate the m/z of the ion.

One consideration in this type of mass analysis is that the set of ions being studied be introduced into the analyzer at the same time. For example, this type of mass analysis is well suited to ionization techniques like MALDI, which produces ions in short well-defined pulses. Another consideration is to control velocity spread produced by ions that have variations in their amounts of kinetic energy. The use of longer flight tubes, ion reflectors, or higher accelerating voltages can help minimize the effects of velocity spread. Time-of-flight mass analyzers have a high level of sensitivity and a wider m/z range than quadrupole or ion trap mass analyzers. Also data can be acquired quickly with this type of mass analyzer because no scanning of the mass analyzer is necessary.

Gas chromatography mass spectrometry offers a nice solution for detecting a target in real-time. The gas chromatography (GC) portion of the system separates the chemical mixture into pulses of analyte and the mass spectrometer (MS) identifies and quantifies the analyte.

Tandem mass spectrometry can utilize combinations of the mass analyzers described above. Tandem mass spectrometers can use a first mass analyzer to separate ions according to their m/z in order to isolate an ion of interest for further analysis. The isolated ion of interest is then broken into fragment ions (called collisionally activated dissociation or collisionally induced dissociation) and the fragment ions are analyzed by the second mass analyzer. These types of tandem mass spectrometer systems are called tandem in space systems because the two mass analyzers are separated in space, usually by a collision cell. Tandem mass spectrometer systems also include tandem in time systems where one mass analyzer is used, however the mass analyzer is used sequentially to isolate an ion, induce fragmentation, and then perform mass analysis.

Mass spectrometers in the tandem in space category have more than one mass analyzer. For example, a tandem quadrupole mass spectrometer system can have a first quadrupole mass filter, followed by a collision cell, followed by a second quadrupole mass filter and then the detector. Another arrangement is to use a quadrupole mass filter for the first mass analyzer and a time-of-flight mass analyzer for the second mass analyzer with a collision cell separating the two mass analyzers. Other tandem systems are known in the art including reflectron-time-of-flight, tandem sector and sector-quadrupole mass spectrometry.

Mass spectrometers in the tandem in time category have one mass analyzer that performs different functions at different times. For example, an ion trap mass spectrometer can be used to trap ions of all m/z. A series of rf scan functions are applied which ejects ions of all m/z from the trap except the m/z of ions of interest. After the m/z of interest has been isolated, an rf pulse is applied to produce collisions with gas molecules in the trap to induce fragmentation of the ions. Then the m/z values of the fragmented ions are measured by the mass analyzer. Ion cyclotron resonance instruments, also known as Fourier transform mass spectrometers, are an example of tandem-in-time systems.

Several types of tandem mass spectrometry experiments can be performed by controlling the ions that are selected in each stage of the experiment. The different types of experiments utilize different modes of operation, sometimes called “scans,” of the mass analyzers. In a first example, called a mass spectrum scan, the first mass analyzer and the collision cell transmit all ions for mass analysis into the second mass analyzer. In a second example, called a product ion scan, the ions of interest are mass-selected in the first mass analyzer and then fragmented in the collision cell. The ions formed are then mass analyzed by scanning the second mass analyzer. In a third example, called a precursor ion scan, the first mass analyzer is scanned to sequentially transmit the mass analyzed ions into the collision cell for fragmentation. The second mass analyzer mass-selects the product ion of interest for transmission to the detector. Therefore, the detector signal is the result of all precursor ions that can be fragmented into a common product ion. Other experimental formats include neutral loss scans where a constant mass difference is accounted for in the mass scans. The use of these different tandem mass spectrometry scan procedures can be advantageous when large sets of reporter tags are measured in a single experiment as with multiplex experiments. In typical applications, the amount of PIRT generated during the PCR reaction is determined based on cycle threshold (Ct) value, which represents the number of cycles required to generate a detectable amount of nucleic acid. Determination of Ct values is well known in the art. Briefly, during PCR, as the amount of formed amplicon increases, the signal intensity increases to a measurable level and reaches a plateau in later cycles when the reaction enters into a non-logarithmic phase. By plotting signal intensity versus the cycle number during the logarithmic phase of the reaction, the specific cycle at which a measurable signal is obtained can be deduced and used to calculate the quantity of the target before the start of the PCR. Exemplary methods of determining Ct are described in, e.g., Heid et al. Genome Methods 6:986-94, 1996, with reference to hydrolysis probes.

### EXAMPLES

### Example 1 Principle of the Invention

### Summary

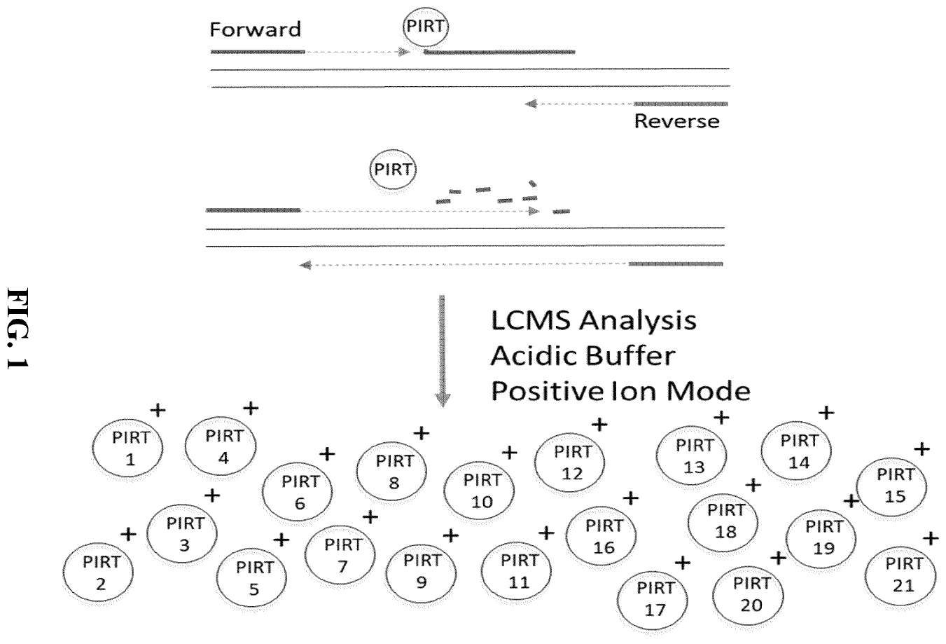

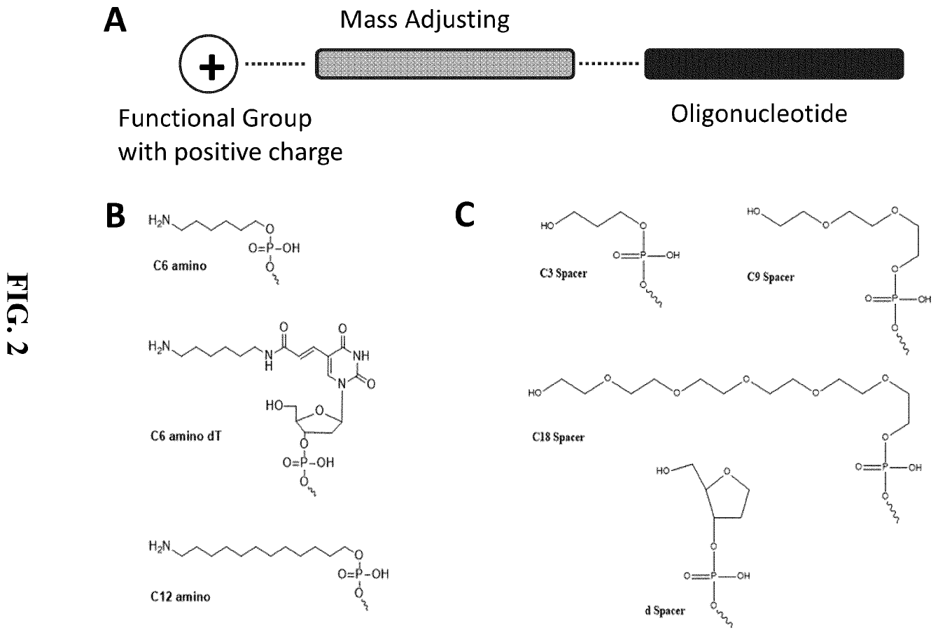

A novel method for detection of multiple PCR targets in a single reaction is provided. This invention utilizes standard TaqMan® PCR chemistry, in which the 5-3′ nuclease activity of a DNA polymerase cleaves a target specific label on a hybridized probe. In this case, the label is a molecule that becomes positively charged under acidic conditions and is detected with positive ion ESI-LCMS (electrospray ionization liquid chromatography mass spectrometer). These Positive Ion Reporter Tags (PIRTs) are small molecules with discrete masses used to identify numerous amplicons similar to how different fluorescent dyes are utilized in TaqMan® technology. The advantage is the facile design of a large number of unique PIRT labels, whereas TaqMan® probe designs are constrained by the limited number of fluorescent dyes available as reporters. The number of distinct molecules that could potentially be used as PIRT labels are so numerous that the number of detectable probes would be un-limiting, thus greatly expanding the multiplexing capability. A graphical representation of the present invention is shown in FIG. 1.

### Background

PCR is a well-established technology that enables the identification of diseases at a genetic level. There are numerous commercialized assays available for the detection of infectious diseases and important biomarkers. The majority of these use TaqMan® chemistry for detection. Briefly, this technique requires a target specific probe and a DNA polymerase with 5-3′ exonuclease activity. The probe contains a fluorophore and a molecule that quenches the fluorescence in its intact native form. The fluorescent signal is revealed when the 5-3′ exonuclease activity of the DNA polymerase cleaves the hybridized probe, liberating the fluorophore from the quencher. Visualization of these fluorescent signals requires expensive and sensitive fluorescence detection systems. The number of detectable targets is limited to the number of dyes that can be differentiated in the visible spectrum.

Mass spectrometry has emerged as a post-PCR detection system with the potential to detect innumerable targets. Agena Bioscience uses the iPLEX chemistry for SNP polymorphism detection. Agilent/Qiagen uses the MassCode PCR technology to identify specific amplicons. Abbott uses the TIGER technology for microbial identification (discontinued in 2017). All these methods require the burden of post PCR sample manipulation and amplicon purification prior to analysis by a mass spectrometer. Nevertheless, they offer an advance in the ability to address the challenges of diagnostics for the many types of infectious agents and syndromes present in the healthcare setting.

PCR ESI-LCMS with PIRT probes combines the strengths of TaqMan® chemistry with the high multiplexing capability afforded by mass spectrometry detection. PIRT probes employ labels that are cheap, readily available, and easy to manufacture. Furthermore, PIRT probes are directly detected, requiring no post-PCR pre-treatment or purification. Acidic LC conditions easily separates PIRT labels from other positively charged molecules, while neutralizing all nucleic acids and dNTPs, rendering them undetectable by the mass spectrometer. This technology has the potential to improve TaqMan® technology by substantially increasing the number of simultaneously detectable targets.

### Description

Positive Ion Reporter Tag (PIRT) probes are labeled on the 5′-end with a chemical modification, such as an amino group, that is protonated under acidic conditions. Amine-modified probes can typically be generated during oligonucleotide synthesis whereby the amino group is attached to the 5′ terminus nucleotide via linkage to carbon chains that have various lengths. Such carbon chains are available as C3, C6, C9, C12 and C18 spacer phosphoramidites and are commercially available (for example at Glen Research, Sterling, VA and Integrated DNA Technologies, Inc. Coralville, IA). Furthermore, additional positive ion tags having unique masses can be generated by adjusting the mass with the addition of the spacers as neutral entities. Non-limiting examples of positive ion tags are shown in FIG. 9. Probe sequences are designed to target specific amplicons in the same fashion as TaqMan® probes. Standard PCR conditions are used with a DNA polymerase having 5-3′ exonuclease activity, such as Taq polymerase. The DNA polymerase cleaves the label during standard PCR thermal cycling, leaving the PIRT itself or the PIRT attached to some number of DNA bases, depending on the cleavage efficiency. The completed PCR reactions are transferred to a LCMS and directly sampled into the system (see FIG. 1).

The LCMS system minimally consists of a binary HPLC, an autosampler and an electrospray ionization mass spectrometer in positive ion mode. The HPLC utilizes a reverse phase column and an acidic buffer system with an organic solvent for elution. Preferred acidic buffers include but are not limited to formic acid, acetic acid and trifluoroacetic acid that have a pH of less than 4.0, preferably between pH 2.0 and pH 4.0. Among the reaction molecules detected under these conditions are buffer components, PIRT labels, PIRT intact probes, detergents and proteins such as DNA polymerase and UNG. The chromatography method is optimized such that the buffer components elute in the column void volume, while PIRT labels elute in a gradient followed by a wash step to elute all other constituents. The target sequences present are identified by analysis of the mass spectrum for the target mass of each PIRT label. The target mass is pre-determined in single-plex reactions to identify the cleavage fragments. The most abundant fragment(s) are selected as the mass for detection of target sequence.

### Example 2 Design of PIRTs

The Positive Ion Reporter Tag (PIRT) is designed with a positively charged group and may include the addition of a mass adjusting subunit (FIG. 2A). The PIRT can be coupled to the 5′-terminus of the oligonucleotide probe during automated DNA synthesis (e.g. by solid-phase phosphoramidite synthesis method). Examples of positively charged functional groups include a phosphorylated carbon chain attached to an amino group (see FIG. 2B, C6-amino and C12-amino moieties) or a nucleotide attached with an amino modified carbon chain (see FIG. 2B, C6-amino-deoxythymidine (dT) moiety). Various neutral molecules may be added to the same positively charged moiety to serve as a mass adjusting subunit to produce PIRTs having distinguishable masses that can be identified by mass spectrometry. Spacer phosphoramidites (examples shown in FIG. 2C) are commercially available molecules that can serve as mass adjusting subunits.

### Example 3 PCR Experimental Conditions

Real-time PCR experiments were performed using either the cobas® 4800 system or the cobas® 6800/8800 systems platforms (Roche Molecular Systems, Inc., Pleasanton, CA). The final concentrations of the amplification reagents are shown below:

TABLE I
PCR Amplification Reagents

| Master Mix Component | Final Conc (50 uL) | |
|---|---|---|
| DMSO | 5.4 | % |
| NaN3 | 0.027 | % |
| Potassium acetate | 120.0 | mM |
| Glycerol | 3.0 | % |
| Tween 20 | 0.0168 | % |
| EDTA | 44.0 | μM |
| Tricine pH 8.2 | 60.0 | mM |
| Aptamer | 0.22 | μM |
| UNG Enzyme | 0.2 | U |
| Z05 DNA Polymerase | 0.9 | U/μl |
| dNTP mix (total) | 2.0 | mM |
| Forward primer oligonucleotides | 0.40 | μM |
| Reverse primer oligonucleotides | 0.40 | μM |
| PIRT Probe oligonucleotides | 0.20 | μM |
| Manganese Acetate | 3.30 | mM |

The following table shows the typical thermal profile used for PCR amplification reaction, whereby PCR cycling was divided into two measurements, wherein both measurements apply a one-step setup (combining annealing and extension).

TABLE II
PCR Thermoprofile

| | Target | Acquisition | Hold | Ramp Rate | | |
|---|---|---|---|---|---|---|
| Program Name | (° C.) | Mode | (hh:mm:ss) | (° C./s) | Cycles | Analysis Mode |
| 1^{st} Measurement | 94 | None | 00:00:15 | 4.4 | 5 | Quantification |
| | 61 | Single | 00:00:30 | 2.2 | | |
| 2^{nd} Measurement | 92 | None | 00:00:15 | 4.4 | 45 | Quantification |
| | 61 | Single | 00:00:30 | 2.2 | | |
| Cooling | 37 | None | 00:02:00 | 2.2 | 1 | None |

### Example 4 Liquid Chromatography Mass Spectrometer (LCMS) Conditions

Detection of PIRT labels post-PCR was performed as follows. Individual reactions were transferred from PCR plate wells into autosampler vials with no required downstream processing. The amplicons were analyzed directly on an LCMS consisting of an Agilent 1260 HPLC and 6530 QTOF mass spectrometer with a dual ESI spray chamber. Chromatography consisted of a Waters XBridge Oligonucleotide BEH C18 column (2.1×50 mm, 130 Å, 2.5μm) with 0.1% formic acid as mobile phase A and methanol as mobile phase B. A linear gradient of 5-80% mobile phase B was run over 14 minutes at a flow rate of 0.3 ml/min. The column was washed with 100% mobile phase B for 2 minutes and re-equilibrated for 5 minutes. The injection volume was 35 μl. The mass spectrometer polarity was positive, with Nitrogen gas temperature of 350° C. and a flow rate of 12 L/min. The Nebulizer pressure was 35 psi, Fragmentor voltage was 200V and Capillary voltage was set at 4000V. The acquisition range was 100-2000 m/z. The following table summarizes the parameters and conditions for performing LCMS.

TABLE III
LCMS parameters

| Column: | Waters Xbridge Oligonucleotide BEH C18 | | |
|---|---|---|---|
| | 2.1 × 50 mm, 130 A, 2.5 μm | | CV = 0.12 ml |
| | P/N | 1.86E+08 | |
| | S/N | 01733834614520 | |
| | L/N | 0173383461 | |
| MP A: | 0.1% Formic Acid | | |
| MP B: | Methanol | | |
| Flow: | 0.3 ml/min | | |
| Min | % B | | |
| 0 | 5 | | Slope: 7.5 |
| 14 | 80 | | |
| 14.5 | 100 | | |
| 16.5 | 100 | | |
| 17 | 5 | | |
| 20 | 5 | | Post time: 2 min |

| Polarity: | Pos | |
|---|---|---|
| N2 T: | 350 | C. |
| N2 Flow: | 12 | L/min |
| Nebulizer: | 35 | psi |
| VFrag: | 200 | V |
| Vcap: | 4000 | V |
| Acq | 100-2000 | |
| Ref | 119,922 | |
| Inj | 35 | μl (neat reaction) |

### Example 5 Synthesis of PIRT Probes

TaqMan® oligonucleotide probes previously used in a real-time PCR assay for detecting various subtypes of the Human Papillomavirus (HPV) were re-designed by removing the reporter and quencher dyes and replacing them with PIRT labels. The HPV probes targeting HPV subtypes 33, 16 and 18 were synthesized by a commercial vendor (Integrated DNA Technologies, Inc.) to have an amino modified 6-carbon deoxythymidne (AmC6dT) PIRT label, an amino modified 6-carbon chain (AmC6) PIRT label, and an amino modified 12-carbon chain (AmC12) PIRT label, respectively, all at the 5′-terminus. Also, a generic internal control (GIC) PIRT probe was synthesized using an amino modified 6-carbon deoxythymidine plus a 3-carbon spacer as a mass adjuster (SpC3/AmC6dT). FIG. 3 shows the structures of the PIRT labels and the oligonucleotide sequences of the PIRT probes that target GIC, HPV 33, 16 and 18.

### Example 6 LCMS Detection of PIRT Label Released during PCR Amplification of Target

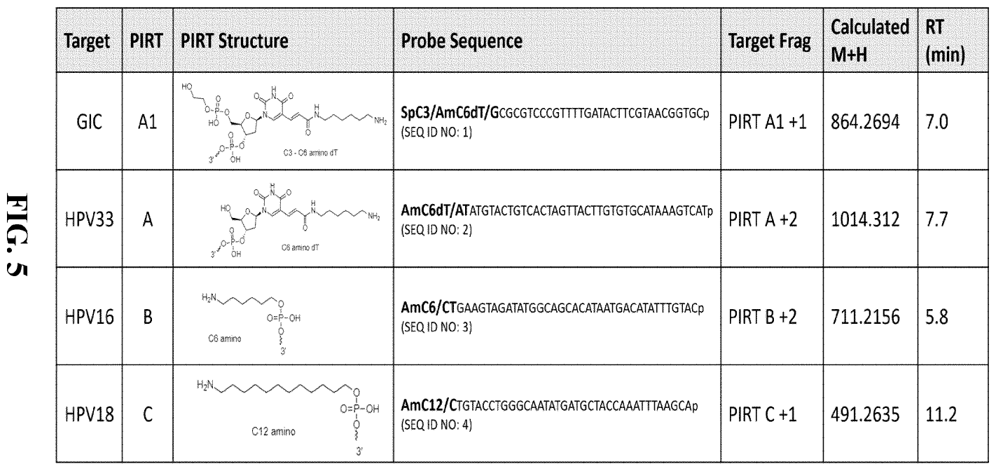

While the PIRT tag is liberated by the 5-3′ exonuclease activity of the DNA polymerase during TaqMan® PCR amplification of the targeted sequence, the cleaved fragment may include a few of the terminal nucleic acid bases from the probe depending on the polymerase cleavage efficiency. Therefore, the mass of the cleaved PIRT tag must be individually determined for each PIRT probe. The PIRT probe targeting HPV 16 has an amino label on a 6-carbon chain that is attached with a phosphodiester linkage to the 5′-terminus of the oligonucleotide probe (referred as PIRT B-HPV16 Probe in FIG. 4A). Using the conditions described in Example 3, a single-plex TaqMan PCR assay was performed with this PIRT B-HPV16 Probe to amplify HPV 16 DNA. The cleaved PIRT tag was then detected by LCMS using the conditions described in Example 4. Upon analysis of the mass spectrum (FIG. 4B), it was shown that the majority of the cleaved fragment generated a peak with an exact mass-to-charge (m/z) of 711.2148. This molecular weight corresponded to the protonated (“H) form of the PIRT B label with the addition of 2 terminal nucleotides (CT) from the 5′ end of the probe, which would result in a calculated exact m/z of 711.2156 (seen in FIG. 4A). The m/z of this protonated cleavage fragment can then be plotted against retention time in the construction of an extracted ion chromatogram (EIC) as shown in FIG. 4C. For the PIRT B-HPV 16 Probe, the retention time was calculated to be approximately 5.8 minutes. The value of the EIC peak and its retention time are then used to identify the amplification and detection of the target nucleic acid.

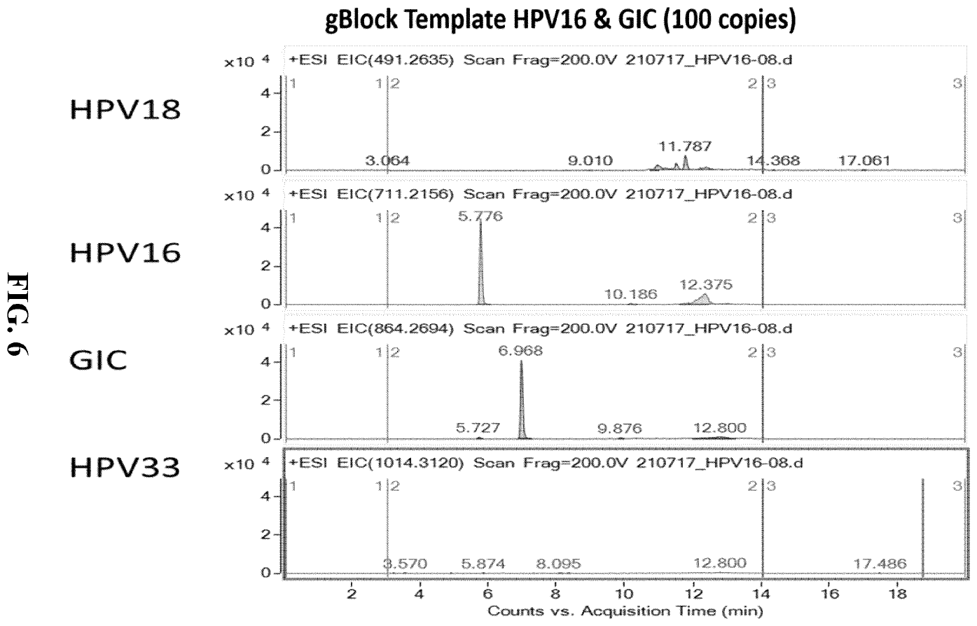

Similar single-plex TaqMan® PCR assays were performed with the PIRT A1-GIC Probe, the PIRT A-HPV33 Probe and the PIRT C-HPV18 Probe to determine the exact m/z of the most abundant fragment for each probe and the retention time. The results are displayed in FIG. 5 and show a retention time of 7.0 minutes, 7.7 minutes and 11.2 minutes for the GIC Probe, HPV 33 Probe and HPV 18 Probe, respectively.

### Example 7 Multiplex PCR Detection of HPV Subtypes Using PIRT Probes

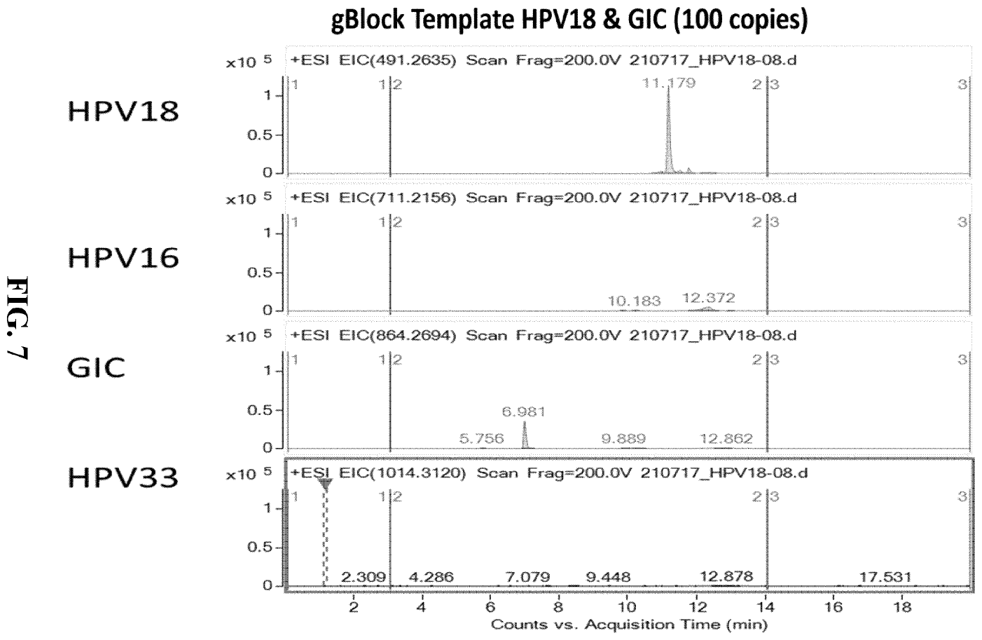

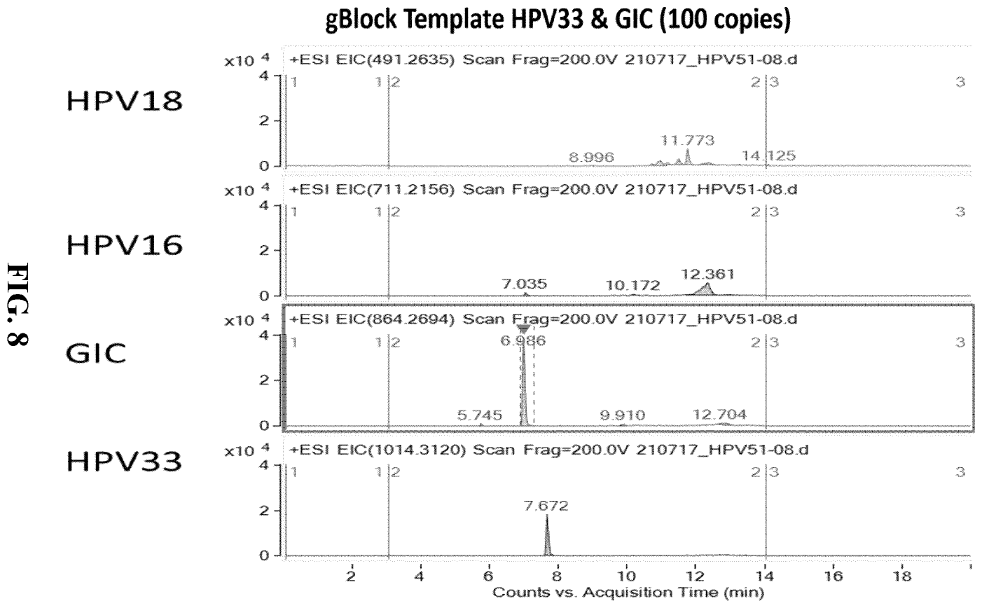

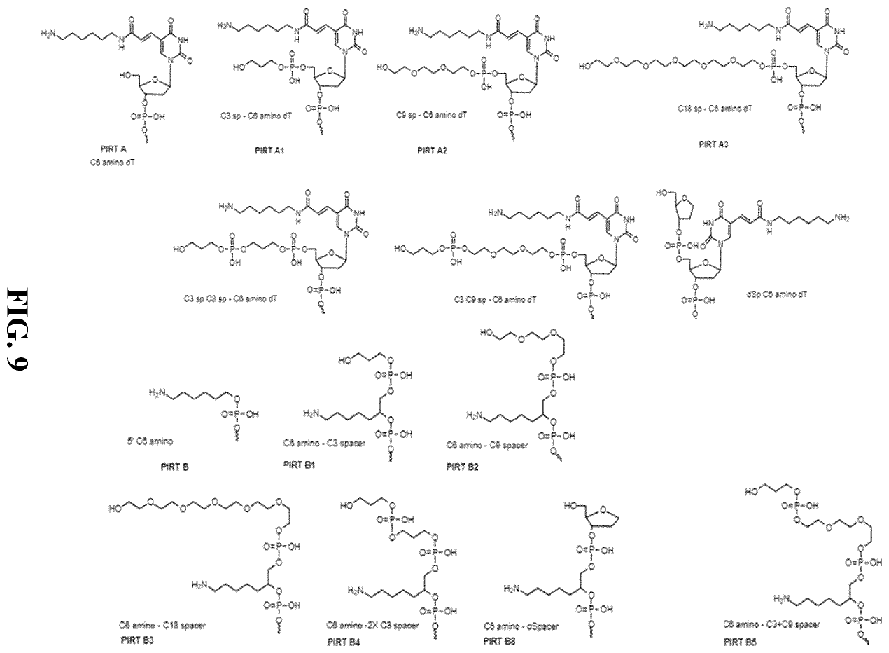

PCR multiplex reaction was set up for amplification and detection of the three HPV targets (subtypes 16, 18 and 33) and a generic internal control (GIC). The master-mix included 0.9 U/μl Z05 DNA polymerase, 400 nM of forward and reverse primers and 200 nM of PIRT labeled probes for each of the four targets. A 25 μl aliquot of the common master-mix was mixed with three sets of 25 μl of a specific target and GIC-synthetic double stranded DNA (gBlock DNA) as template, with the first set containing HPV16 and GIC, the second set containing HPV 18 and GIC, and the third set containing HPV 33 and GIC. The PCR reactions were transferred to autosampler vials and analyzed directly by LCMS with no sample preparation necessary. Extracted ion chromatograms (EIC) were then constructed for each of the four target masses, in which the presence of a peak indicates the positive detection of the corresponding target. The EIC generated from the first, second and third sets are shown in FIGS. 6, 7 and 8, respectively. In FIG. 6, peaks are detected only in the HPV 16 and GIC masses indicating the amplification and presence of these targets. As expected, no peaks at the corresponding retention time (RT) were detected for the HPV 18 and HPV 33 masses, indicating the absence of these targets in this reaction. Similarly, in FIG. 7, peaks were observed only in the EIC for HPV 18 and GIC and in FIG. 8, peaks were seen for HPV 33 and GIC only. Small late eluting non-target specific peaks were observed in some chromatograms, which can be removed by further assay optimization.

1. A method of detecting the presence or absence of a target nucleic acid sequence in a sample, comprising the steps of:
(a) contacting a sample comprising a target nucleic acid with
(i) a pair of oligonucleotide primers comprising a first oligonucleotide primer and a second oligonucleotide primer, wherein the first oligonucleotide primer comprises a sequence complementary to a region in one strand of the target nucleic acid sequence and is capable of priming the synthesis of a first extension product, and wherein the second oligonucleotide primer comprises a sequence complementary to a region in said first extension product and is capable of priming the synthesis of a nucleic acid strand complementary to said first extension product, and
(ii) an oligonucleotide probe comprising a Positive Ion Reporter Tag (PIRT) at the 5′-terminus, whereby the PIRT is protonated under acidic conditions;
(b) amplifying said target nucleic acid sequence with a nucleic acid polymerase having 5′ to 3′ nuclease activity under conditions that allow annealing of said pair of oligonucleotide primers and said oligonucleotide probe to the target nucleic acid sequence and synthesis of primer extension products from said pair of oligonucleotide primers, while the 5′ to 3′ nuclease activity of said nucleic acid polymerase is able to cleave and release from the annealed oligonucleotide probe fragments containing the PIRT with or without additional nucleotides from the oligonucleotide probe; and
(c) detecting the presence or absence of the PIRT by an electrospray ionization liquid chromatography mass spectrometer (ESI-LCMS) under acidic conditions, thereby detecting the presence or absence of the target nucleic acid sequence in the sample;

2. The method of claim 1, wherein two or more target nucleic acids are detected in a single multiplexed reaction.

3. The method of claim 2, wherein two or more oligonucleotide probes are used to detect the two or more target nucleic acids in the single multiplexed reaction.

4. The method of claim 1, wherein the amplifying step is a polymerase chain reaction (PCR) or a real-time polymerase chain reaction (RT-PCR).

5. The method of claim 1, wherein the detecting step (c) is done by a mass spectrometer, selected from Electrospray Ionization-Time of Flight Mass Spectrometer (ESI-TOF-MS) or Electrospray Ionization-Liquid Chromatography Mass Spectrometer (ESI-LC-MS).

6. The method of claim 1, wherein the nucleic acid polymerase is a thermostable DNA polymerase.

7. The method of claim 1, wherein the PIRT comprises a positive ion entity selected from C6-amino dT, C6-amino, and C12-amino.

8. The method of claim 7, wherein the PIRT further comprises spacer moieties selected from C3 Spacer, C9 Spacer, C18 Spacer, d Spacer and combinations of the spacer moieties thereof.

9. A composition comprising an oligonucleotide probe, wherein said oligonucleotide probe comprises a Positive Ion Reporter Tag (PIRT) at the 5′-terminus, wherein the PIRT is protonated under acidic conditions.

10. The composition of claim 9, wherein the PIRT comprises a positive ion entity selected from C6-amino dT, C6-amino, and C12-amino.

11. The composition of claim 10, wherein the PIRT further comprises spacer moieties selected from C3 Spacer, C9 Spacer, C18 Spacer, d Spacer, and combinations of the spacer moieties thereof.

12. The method of claim 1, wherein no post-amplification sample purification step is performed between step (b) and step (c).

13. A method of detecting the presence or absence of at least two target nucleic acid sequences in a sample, comprising the steps of:
(a) contacting a sample comprising at least a first target nucleic acid and a second target nucleic acid with
(i) a first pair of oligonucleotide primers corresponding to the first target nucleic acid, the first pair of primers comprising a first oligonucleotide primer and a second oligonucleotide primer, wherein the first oligonucleotide primer comprises a sequence complementary to a region in one strand of the first target nucleic acid sequence and is capable of priming the synthesis of a first extension product, and wherein the second oligonucleotide primer comprises a sequence complementary to a region in the first extension product and is capable of priming the synthesis of a nucleic acid strand complementary to the first extension product,
(ii) a second pair of oligonucleotide primers corresponding to the second target nucleic acid, the second pair of primers comprising a third oligonucleotide primer and a fourth oligonucleotide primer, wherein the third oligonucleotide primer comprises a sequence complementary to a region in one strand of the second target nucleic acid sequence and is capable of priming the synthesis of a second extension product, and wherein the fourth oligonucleotide primer comprises a sequence complementary to a region in the second extension product and is capable of priming the synthesis of a nucleic acid strand complementary to the second extension product, and
(iii) a first oligonucleotide probe comprising a first Positive Ion Reporter Tag (PIRT) at the 5′-terminus, and a second oligonucleotide probe comprising a second PIRT at the 5′-terminus, whereby the first and second PIRT are each protonated under acidic conditions;
(b) amplifying the first and second target nucleic acid sequences with a nucleic acid polymerase having 5′ to 3′ nuclease activity under conditions that allow
(i) annealing of the first pair of oligonucleotide primers and the first oligonucleotide probe to the first target nucleic acid sequence and synthesis of primer extension products from the first pair of oligonucleotide primers, and
(ii) annealing of the second pair of oligonucleotide primers and the second oligonucleotide probe to the second target nucleic acid sequence and synthesis of primer extension products from the second pair of oligonucleotide primers,
wherein the 5′ to 3′ nuclease activity of the nucleic acid polymerase is able to cleave and release from the annealed first and second oligonucleotide probe fragments containing the first and second PIRT with or without additional nucleotides from the oligonucleotide probe; and
(c) detecting the presence or absence of the first and second PIRT by an electrospray ionization liquid chromatography mass spectrometer (ESI-LCMS) under acidic conditions, thereby detecting the presence or absence of the first and second target nucleic acid sequences in the sample.

14. The method of claim 13, wherein no post-amplification sample purification step is performed between step (b) and step (c).

15. The method of claim 13, wherein the first and second PIRT each comprise a positive ion entity selected from C6-amino dT, C6-amino, and C12-amino.

16. The method of claim 15, wherein the first and second PIRT each further comprise spacer moieties selected from C3 Spacer, C9 Spacer, C18 Spacer, d Spacer and combinations of the spacer moieties thereof.


SEQUENCE LISTING (4 sequences)

SEQ ID NO: 1 - DNA, 32 bp, synthetic construct
  misc_feature 1..32: note = PIRT Probe GIC
  misc_feature 1..32: note = 5` SpC3/AmC6dT
  misc_feature 1..32: note = 3` phosphate
  source 1..32: mol_type = other DNA
gcgcgtcccgttttgatacttcgtaacggtgc

SEQ ID NO: 2 - DNA, 39 bp, synthetic construct
  misc_feature 1..39: note = PIRT Probe HPV33
  misc_feature 1..39: note = 5` AmC6dT
  misc_feature 1..39: note = 3` phosphate
  source 1..39: mol_type = other DNA
atatgtactgtcactagttacttgtgtgcataaagtcat

SEQ ID NO: 3 - DNA, 39 bp, synthetic construct
  misc_feature 1..39: note = PIRT Probe HPV16
  misc_feature 1..39: note = 5` AmC6
  misc_feature 1..39: note = 3` phosphate
  source 1..39: mol_type = other DNA
ctgaagtagatatggcagcacataatgacatatttgtac

SEQ ID NO: 4 - DNA, 37 bp, synthetic construct
  misc_feature 1..37: note = PIRT Probe HPV18
  misc_feature 1..37: note = 5` AmC12
  misc_feature 1..37: note = 3` phosphate
  source 1..37: mol_type = other DNA
ctgtacctgggcaatatgatgctaccaaatttaagca